(19) United States
(12) Patent Application Publication
(10) Pub. No.: US 2005/0027241 A1
(43) Pub. Date: Feb. 3, 2005

(54) Wound and eye irrigator

(76) Inventor: Rourke M. Yeakley, Eagle, ID (US)

Correspondence Address: FRANK J. DYKAS;DYKAS, SHAVER & NIPPER, LLP, PO BOX 877, BOISE, ID, 83701-0877, US

(21) Appl. No.: 10/632,874

(22) Filed: Jul. 30, 2003

(51) Int. Cl. A61M 31/00

(52) U.S. Cl. 604/93.01; 604/298

(57) ABSTRACT

A portable medical irrigation device made up of a container configured to hold a designated quantity of a desired irrigating liquid therein. The container has a reservoir that holds a quantity of an irrigating liquid, a hand operated pump, and a conduit connected to the pump. The pump is configured to transfer irrigation liquid from the reservoir to an area to be irrigated through the conduit. The conduit has a nozzle end configured to allow a user to select between a stream and a spray delivery pattern. The nozzle end also contains a fluid deflector that in combination with the nozzle end deliver irrigating liquid of a designated quantity and character to a designated location.

### BACKGROUND OF THE INVENTION

Field of the Invention. The present invention generally relates to an apparatus for irrigating wounds and eyes, and more particularly to a self-contained portable apparatus for irrigating wounds and eyes in a either a hospital or non-hospital setting.

Background Information. Irrigation of wounds and eyes is a delicate and sensitive procedure, which can in many instances be the difference between permanent damage and full recovery. When an individual is wounded, the wound typically breaks the surface of a person's skin and exposes portions of the body to a variety of pathogenic materials. These materials can cause great damage to the wounded area. However, in many instances the same bacteria that cause these diseases can also oftentimes be easily removed from the wounded area by irrigating or flushing the area with substances such as water or simple saline. Irrigating the area acts to prevent future damage to the affected area by removing the materials from the affected area.

In addition to removing pathogenic bacteria from a wounded area, irrigation devices may also be used to remove caustic and/or other types of damaging chemical agents from contact in the affected area, such as so called splash injuries that occur when a substance is splashed upon an individual.

Irrigation of eyes and wounds can alleviate the symptoms associated with many disturbances which would otherwise plague a person in a non-hospital setting. For example substances such as wind, dust and sun can cause damage to an individual's skin and eyes. Irrigation of these areas with a liquid such as water or saline may help to moisten these areas, remove damaging material and prevent or limit such future damage from occurring. In order to properly flush or irrigate an area of a person's body, care must be exercised. On the one hand a sufficient amount of pressure must be applied to the rinsing liquid so as to displace the foreign materials from the area. On the other hand, the amount of pressure must not be so great that damage to the tissues, particularly the already damaged tissues of the individual's body takes place.

One problem that may occur is that while in some instances simply pouring a quantity of water or other solution over the affected area may not produce sufficient pressure to remove the foreign material from the affected area. Additionally, in remote locations, access to additional stores of proper irrigation liquids cannot be obtained. Therefore, the irrigation devices must be specifically crafted to deliver sufficient quantities of irrigation materials under a proper amount of pressure in a way that adequately provides the desired advantages, and which allows the user to consistently have a sufficient amount of irrigating materials on hand. Another requirement is that an irrigating device and solution be compact and easy to use in a situation such as a non-hospital setting. Another requirement is that the present devices also be easy to use by persons with limited or no medical training.

Accordingly it is an object of the present invention to provide a portable wound and eye irrigation system and device that is configured to provide designated quantities of irrigation liquid at proper pressures to designated portions of a person's body and to flush areas such as wounds. It is another object of the invention to provide a designated quantity of an irrigating liquid under a properly designated amount of pressure so as to properly enable a person utilizing the device to properly irrigate an area without causing damage to the area being irrigated. Furthermore, the present invention has an object of being a self-contained irrigating device that can provide a variety of differing desired amounts of pressure to an individual and which can be further utilized to use irrigating fluids in a proper and efficient manner.

The present invention provides a self-contained cleansing irrigating device, which can be utilized to clean infected portions of a person's body in a non-hospital setting. The present invention is self-contained and provides a variety of levels of irrigating pressures so as to allow an individual to vary the desired amount of pressure that is delivered to an area being irrigated. The present invention also provides an irrigating device that provides simple, effective medical irrigation in a portable or outdoor setting.

Additional objects, advantages and novel features of the invention will be set forth in part in the description which follows and in part will become apparent to those skilled in the art upon examination of the following or may be learned by practice of the invention. The objects and advantages of the invention may be realized and attained by means of the instrumentalities and combinations particularly pointed out in the appended claims.

### SUMMARY OF THE INVENTION

A portable medical irrigation device for irrigation of affected areas comprising a container configured to hold a designated quantity of a desired irrigating liquid therein. The container having a reservoir configured to hold a designated quantity of the irrigating liquid, a pump, and a conduit connected to the pump. The pump configured to transfer the irrigation liquid from the reservoir to an area to be irrigated through the conduit. The conduit also has a nozzle end. The nozzle end configured to deliver the irrigating liquid from the reservoir to the affected area.

In the preferred embodiment, the irrigating liquid is normal saline that is transferred to the nozzle end by a pump. The pump is configured to operatively draw irrigating liquid out of the reservoir and to deliver the irrigating liquid to a site to be irrigated. Variations in the velocity and pressure of the irrigating solution being delivered to the area can be modified by varying the force and frequency at which the pump is activated. While in the preferred embodiment the pump is a hand pump it is to be distinctly understood that the invention is not limited thereto but may be variously embodied to include a variety of other types of features, this includes battery operated pumping devices. The pump pumps the liquid irrigating material out of the reservoir through conduit and is configured to deliver the material upon a desired location. This delivery is assisted by a nozzle end that is configured to provide a alternatively a stream having sufficient washing properties so as to irrigate a targeted area, a dispersing spray for lavaging areas such as eyes and mucosal membranes.

The nozzle end is configured to provide a dispersing spray for irrigating eyes, as well as a more concentrated stream for cleaning other parts of an individual's body and other types of wounds. In the preferred embodiment, the nozzle end is reconfigurable so as to allow alternating change between these two types of discharges, in addition a fluid deflector assists to focus the irrigating liquid upon the appropriate desired location.

In some instances, the portable medical irrigation device of claim 1 is configured for storage within a transportable container. In other embodiments, the portable medical irrigation device is configured to allow removable reservoirs of various types of liquids to be interchanged within the device. In other embodiments the reservoir is refillable through a desired opening, while in some other embodiments the reservoir is not refillable in any way.

In use, the device may be used by simply activating the hand pump portion of the device to deliver irrigating liquid through the conduit to the nozzle end. The nozzle end can then be selectively arranged so as to allow fluid from the device to be placed upon a desired location and to irrigate the device. The present invention is a self-contained unit that can be simply and easily stored and then simply reconfigured to provide a simple field irrigation device and system that is configured for use in a non-hospital setting. The device provides for long term and durable storage of irrigating liquid as well as a delivery device for properly and efficiently delivering the liquid material from this device to a designated body part in a non-hospital setting.

The purpose of the foregoing abstract is to enable the United States Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measure by the claims, nor is it intended to be limiting as to the scope of the invention in any way.

Still other objects and advantages of the present invention will become readily apparent to those skilled in this art from the following detailed description wherein I have shown and described only the preferred embodiment of the invention, simply by way of illustration of the best mode contemplated by carrying out my invention. As will be realized, the invention is capable of modification in various obvious respects all without departing from the invention. Accordingly, the drawings and description of the preferred embodiment are to be regarded as illustrative in nature, and not as restrictive in nature.

### BRIEF DESCRIPTION OF THE DRAWINGS

FIG. 1 a perspective side view of a preferred embodiment of the present invention is shown.

FIG. 2 is a side plan view of an embodiment of the present invention.

### DESCRIPTION OF THE PREFERRED EMBODIMENTS

While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

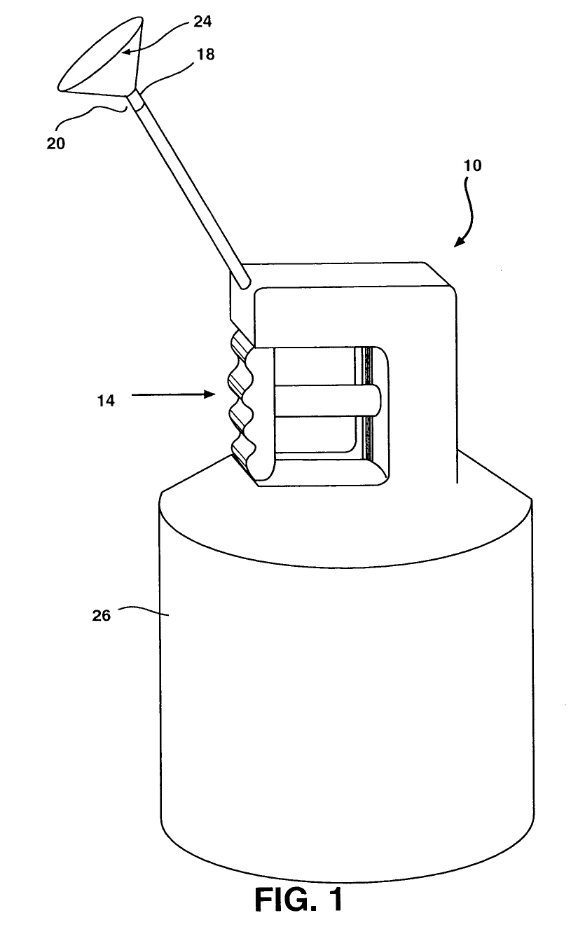

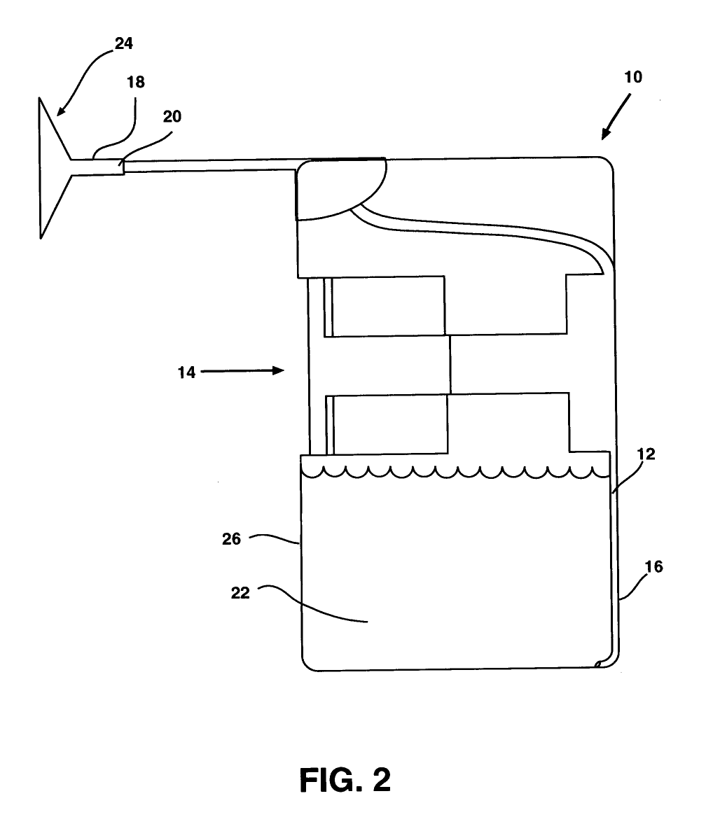

Referring now to FIGS. 1 and 2, several view of a preferred embodiment of the present invention is shown. The present invention is an irrigating device 10 made up of a container 12. The container 12 is made of a resilient material and provides support for a reservoir 26 and a housing for a pump 14. The container 12 is also configured to form a part of the pump 14 and to provide a conduit 16 from the reservoir to the pump. In the preferred embodiment, this reservoir 26 is configured to hold about one half-liter of normal, sterile saline.

While normal sterile saline is shown as the liquid utilized in this preferred embodiment it is to be distinctly understood that the invention is not limited thereto but may be variously embodied to accommodate a variety of necessities of the user. Therefore the type of liquid that is utilized in the device should be seen as illustrative in nature and not as limiting. In some embodiments of the invention, the reservoir is replaceable with interchangeable bags of irrigating solution, while in other embodiments, the reservoir that is provided with the device initially is not replaceable or refillable. However, it is to be distinctly understood that the present invention is not limited to such embodiments, but may be variously embodied to accommodate the needs of the user. This may include a variety of types of irrigating solutions including but not limited solutions having various desired acidic, caustic, and anti-bacterial features.

A conduit 16 extends from the reservoir 26 through a portion of the container 12 from the reservoir 26 to the pump 14. The conduit then also leads from the pump 14 to the nozzle end 18. The conduit 16 is configured to bring irrigation material 22 from the reservoir 26 through the pump 14 to the nozzle end 18. The nozzle end 18 is then configured to disperse the irrigation material 22 upon the intended area.

The pump 14 is configured to pump the irrigation liquid 22 through the conduit 16 to the nozzle end 18. In the preferred embodiment, a hand pump 14 is shown. However, it is to be distinctly understood that the present invention is not limited thereto. In various embodiments of the invention, the pump 14 for pushing irrigation fluid through the conduit 16 to the nozzle end 18 may be a battery operated pump or a pre-pressurized cylinder. The pump itself is included as a portion of the container and thus does not require a separate pump or connection in order to prepare the device for use. Furthermore, the present invention also all provides a container with all of the required portions of the device already present and configured for use. As a result such a device is a significant advantage over prior art device that include a variety of materials that draw from a flexible liquid containing bag.

In use, the irrigating fluid 22, is pumped through the conduit 16 to the nozzle end 18. The hand pump 14 pulls the irrigating material 22 through a portion of the conduit 16 and pushes this material out of the nozzle end portion 18 of the device. The nozzle end 18 has a valve 20, which can be variously configured to control the discharge of the irrigating fluid 22 through the nozzle end 18. In the preferred embodiment, the valve 20 is a twistable valve that is configured to variously modify the flow of fluid to an area to be irrigated. In the preferred embodiment, the valve 20 is configured to provide variation between a fine spray-like misting flow of material out of the nozzle end 18, and a more concentrated stream like spray out of the same nozzle end 18 of the device. In this preferred embodiment, the size of the stream that is pushed through the nozzle end 18 is an eighteen-gauge stream. The nozzle end 18 may also be variously configured to provide a shut off position that will prevent the passage of any portion of the material out of the nozzle end portion of the device.

The device is stored in a prepackaged arrangement and can be quickly assembled when ready for use. By sealing the irrigating solution within the reservoir, the solution can be stored for an extended period of time. When use of the device is desired the components of the device can simply be assembled and used. Such a device provides a variety of advantages over the prior art in that it provides a safe effective method for providing a self-contained irrigating device for irrigating wounds and treating splash injuries that can be safely stored and used in a non-hospital setting including in outdoor situations.

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

1. a portable medical irrigation device comprising:
a container configured to hold a designated quantity of a desired liquid therein said container having a reservoir configured to hold a designated quantity of an irrigating liquid, a pump, and a conduit connected to said pump, said pump configured to transfer said irrigation liquid from said reservoir to an area to be irrigated through said conduit, said conduit having a nozzle end, said nozzle end configured to provide a quantity and character of a discharge from said conduit.

2. The portable medical irrigation device of claim 1 wherein said irrigating liquid is normal saline.

3. The portable medical irrigation device of claim 1 wherein said pump is a hand pump, said hand pump configured to operatively draw irrigating liquid out of said reservoir and to deliver said irrigating liquid to a site to be irrigated.

4. The portable medical irrigation device of claim 1 wherein said nozzle end is configured to provide a stream for irrigating wounds.

5. The portable medical irrigation device of claim 1 wherein said nozzle end is configured to provide a dispersing spray for irrigating eyes.

6. The portable medical irrigation device of claim 1 wherein said nozzle end is configured to allow a user to select between a stream for irrigation and a spray for irrigation.

7. The portable medical irrigation device of claim 1 wherein said nozzle end also contains a fluid deflector.

8. The portable medical irrigation device of claim 1 wherein said container is configured for storage within a transportable container.

9. The portable medical irrigation device of claim 1 wherein said reservoir is configured to removable connection with said container, and wherein a partition separates said reservoir from other portions of said container.

10. The portable medical irrigation device of claim 1 wherein said pump is a battery operated pump.

11. A portable medical irrigation device comprising:
a container configured to hold a designated quantity of a desired irrigating liquid therein said container having a reservoir configured to hold a designated quantity of said irrigating liquid, a hand operated pump, and a conduit connected to said pump, said pump configured to transfer said irrigation liquid from said reservoir to an area to be irrigated through said conduit, said conduit having a nozzle end, said nozzle end configured to allow a user to select between a stream for irrigation and a spray for irrigation, said nozzle end also contains a fluid deflector said nozzle end and fluid deflector configured to deliver liquid of a designated quantity and character to a designated location.
